(19) United States
(12) Patent Application Publication
(10) Pub. No.: US 2012/0101480 A1
(43) Pub. Date: Apr. 26, 2012

(54) CATHETER SHAFT

(75) Inventors: FRANK INGLE, PALO ALTO, CA (US); ROBERT F. BENCINI, SUNNYVALE, CA (US)

(73) Assignee: BOSTON SCIENTIFIC SCIMED, INC., MAPLE GROVE, MN (US)

(21) Appl. No.: 12/909,727

(22) Filed: Oct. 21, 2010

(51) Int. Cl. A61M 25/00; B32B 37/02

(52) U.S. Cl. 604/526; 156/184; 156/190

(57) ABSTRACT

A catheter shaft having a helically wound elongate tubular member is disclosed. The catheter shaft may include a tubular member formed from helically wrapped length of material. The length of material may be wrapped to define a plurality of turns. At least some of the turns may be wrapped in an overlapping fashion such that at least a portion of a first edge of a turn overlaps at least of portion of a second edge of a turn of a previous turn defining an overlap distance.

### TECHNICAL FIELD

The disclosure is directed to medical devices. More particularly, the disclosure is directed to a catheter shaft having a wrapped elongate member.

### BACKGROUND

A variety of elongate tubular shafts for use in medical devices such as catheters, endoscopes, and the like have been developed over the years. There are many known methods of manufacturing an elongate tubular shaft for use in medical devices based on the desired properties of the device. However, it may be desirable to improve the ability to vary the stiffness along the length of an elongate tubular shaft.

### SUMMARY

The disclosure is directed to several alternative designs, materials and methods of manufacturing medical device structures and assemblies.

Accordingly, one illustrative embodiment is a catheter shaft including a tubular member having a proximal end and a distal end. The tubular member may include a length of material helically wrapped defining a plurality of turns. The length of material may have a width including a first edge and a second edge. Some of the turns forming the tubular member may be wrapped in an overlapping fashion such that at least a portion of a first edge of a turn overlaps at least of portion of a second edge of a previous turn defining an overlap distance.

Another illustrative embodiment is a method for manufacturing a medical device having a tubular elongate member. The method may include providing a mandrel having a longitudinal axis, a first end, and a second end and a length of material having a width including a first edge and a second edge. The length of material may be wrapped around the mandrel in a helical manner such that the length of material defines a plurality of turns. At least a portion of the first edge of a turn overlaps at least of portion of the second edge of a previous turn for least some of the turns. The mandrel may be removed such that the plurality of turns defines a tubular member including a lumen.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a plan view of a medical device in accordance with one example embodiment of the invention;

FIG. 2 is a partial plan view of an illustrative catheter shaft having a helically wrapped shaft;

FIGS. 3A and 3B are cross-sections of the illustrative catheter shaft of FIG. 2;

FIG. 4 is a perspective view of an illustrative partially formed catheter shaft having a helically wrapped shaft;

FIG. 5 is an illustrative manufacturing assembly for manufacturing a catheter shaft having a helically wrapped shaft;

FIG. 6 is an illustrative manufacturing assembly for manufacturing a multi-lumen catheter shaft having a helically wrapped shaft;

FIGS. 7A and 7B are perspective views of an illustrative multi-lumen catheter shaft having a helically wrapped shaft;

FIGS. 8A and 8B are cross-sections of the illustrative catheter shaft of FIG. 7A;

FIG. 9A is an illustrative embodiment of a length of material including reinforcing filaments;

FIG. 9B is an illustrative partially formed catheter shaft having variable a helically wrapped shaft;

FIG. 10A is an illustrative embodiment of a length of material including reinforcing filaments;

FIG. 10B is an illustrative partially formed catheter shaft having a helically wrapped shaft;

FIG. 11A is perspective views of an illustrative catheter shaft having a helically wrapped shaft; and

FIG. 11B is perspective views of an illustrative multi-lumen catheter shaft having a helically wrapped shaft.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary. While the embodiments described herein may be described in terms of spatial orientation, the terminology used is not intended to be limiting, but instead to provide a straightforward description the various embodiments.

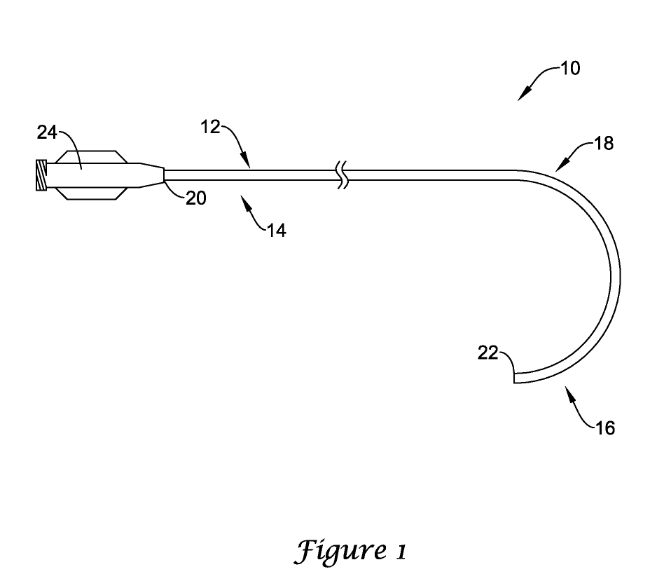

Turning to FIG. 1, which illustrates a medical device 10 in accordance with one example embodiment. In the embodiment shown, the medical device may be in the form of a guide or diagnostic catheter 10. Although set forth with specific reference to a guide or diagnostic catheter, in the example embodiments shown in the Figures and discussed below, the invention may relate to virtually any medical device including an elongate shaft or member. For example, the invention may be applied to medical devices such as a balloon catheter, an atherectomy catheter, a drug delivery catheter, a stent delivery catheter, an endoscope, an introducer sheath, a fluid delivery device, other infusion or aspiration devices, device delivery devices, and the like. Thus, while the Figures and descriptions below are directed toward a guide or diagnostic catheter, in other applications sizes in terms of diameter and length may vary widely, depending upon the desired properties of a particular device. For example, in some devices lengths may range from about 1-300 centimeters (cm) or more, while the outside diameter may range from about 1 French (F) to about 20 F, or even more in some embodiments.

The illustrative catheter 10 may have a length and an outside diameter appropriate for its desired use, for example, to enable intravascular insertion and navigation. For example, the catheter 10 may have a length of about, for example, 5-200 cm, 75-150 cm, or 90-130 cm and an outside diameter of approximately 3-20 F, 5-15 F, or 6-10 F when catheter 10 is adapted as a guide catheter. The illustrative catheter 10 may include structure and materials that are substantially conventional except as described herein and shown the drawings. While catheter 10 is described in terms of intravascular use, in other embodiments the guide or diagnostic catheter may be suited for other uses in the digestive system, soft tissues, or any other use including insertion into an organism for medical uses.

The illustrative catheter 10 may include an elongate shaft 12 having a proximal end region 14 and a distal end region 16 having an intermediate region 18 disposed there between. Elongate shaft 12 may include a lumen (not explicitly shown) extending from a proximal end 20 to a distal end 22 to facilitate, for example, insertion of other medical devices (e.g., guidewires, balloon catheters, etc.) therethrough, and/or to facilitate injection of fluids (e.g., radiopaque dye, saline, drugs, etc.) therethrough. The proximal end 20 of the elongate shaft 12 may be connected to a manifold and/or hub assembly 24 to facilitate connection to other medical devices (e.g. syringe, Y-adapter, etc.) and to provide access to lumen. It is contemplated that in some embodiments the catheter 10 may exclude the lumen, or may include additional devices such as inflation or anchoring members, sensors, optical elements, ablation devices, or the like. In some embodiments the catheter 10 may be significantly shorter and used as an introducer sheath, for example, while in other embodiments the catheter 10 may be adapted for other medical procedures.

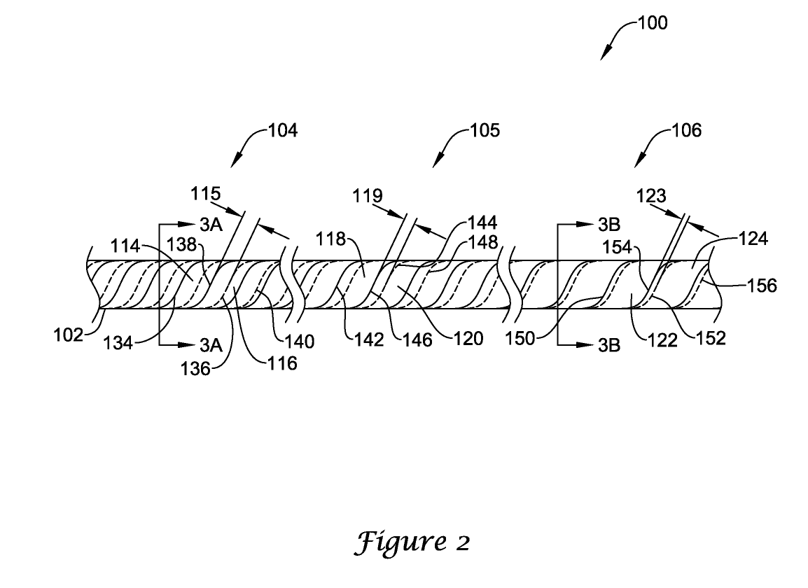

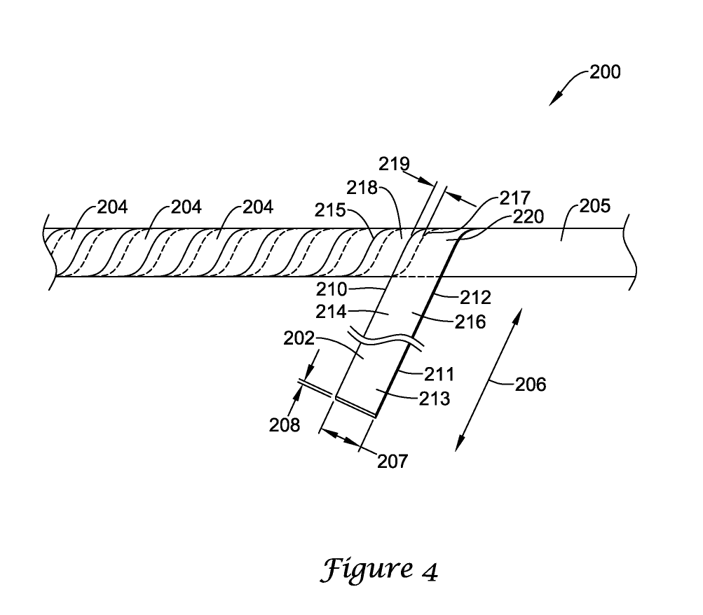

With reference to FIG. 2, a catheter shaft 100 having a helically wrapped elongate tubular member will now be described. As illustrated in FIG. 2, the catheter shaft 100 may be a tubular member 102 formed from a length of material, helically wrapped forming a plurality of turns 114, 116, 118, 120, 122, 124, where every individual turn has not been explicitly identified. In some embodiments, at least some of the turns 114, 116, 118, 120, 122, 124 may be helically wrapped in an overlapping fashion. For the sake of this disclosure, the terminology “turn” or “winding” may be used interchangeably and are both intended to represent a single revolution of the length of material forming the catheter shaft 100. Referring to FIG. 4, the length of material 202 may be a long, thin strip having a width 207 including a first edge, such as proximal edge 210 and a second edge, such as distal edge 212 and a thickness 208 including a outer surface 213 and an inner surface 211. In some embodiments, the width 207 and thickness 208 may both be significantly smaller than the length of the strip of material 202. In some embodiments, the thickness 208 may be significantly smaller than the width 207. The region of the width 207 adjacent to the proximal edge may be the proximal edge region 214 and the region of the width 207 adjacent to the distal edge 212 may be the distal edge region 216. While the length of material 202 is not shown completely from end to end, the length direction is illustrated by arrow 206. The length of material 202 may have a width 207 of about 0.5-10 millimeters (mm), 2-8 mm, or 3-6 mm and a thickness 208 of about 0.005-0.25 mm, 0.01 mm-0.1 mm, or 0.03-0.08 mm. In some embodiments, the length of material 202 may have a width 207 to thickness 208 ratio of about 1000:1, 50:1, or 2:1.

Referring to FIGS. 2 and 4, the length of the strip of the material 202 may vary as a function of the length of the catheter shaft 100, the inner diameter 112 of the catheter shaft 100, the distance of overlap 115, 119, 123 of adjacent turns, and/or the angle of the helical windings, which will be described in more detail below. For example, a longer catheter shaft 100 or a larger inner diameter may require a longer length of material than a shorter catheter shaft 100 or a smaller inner diameter. For a catheter shaft 100 having a number of adjacent turns overlapping, a longer length of material may be needed compared to a catheter shaft 100 having a similar length and inner diameter but no adjacent turns overlapping.

The length of material may comprise any suitable material desired, for example, but not limited to, polymers, metals, or superelastic metal alloys, such as, but not limited to, Kapton®, Mylar®, Polyester, stainless steel, or nitinol. In some embodiments, the material may be considered a “tape”. For example, in some embodiments, the length of material may comprise a strip or sheet of polymeric or metallic material having an adhesive backing. In some instances, the material may be purchased with a pre-applied adhesive. In other instances, the adhesive may be applied during the manufacturing process of the catheter shaft 100. In some embodiments, the adhesive may be applied to only a portion of one side of the length of material such as the portion of the length of material that may overlap a preceding turn. Additionally, the material may be hydrophilic, which may provide strong adhesive bonds when adjacent turns overlap. In some embodiments, the material may have its surface activated by plasma, roughened by, for example, sandpaper, blasting, or peened, or textured with rollers to enhance adhesive bonds when adjacent turns overlap. In some embodiments, the material may comprise Kapton®, a polyimide film, available from DuPont. In other embodiments, the material may comprise Mylar®, a biaxially-oriented polyethylene terephthalate (boPET) available from DuPont. Tape materials such as Kapton® or Mylar® may have a higher tensile strength than extruded materials, such as polyethylene. In some embodiments, the length of material may be cut to the desired dimensions from a larger sheet of material. In other embodiments, the length of material may be cut to a desired length from a stock of material having the desired width and thickness. While not explicitly shown, in other embodiments, the length of material may be formed of a plurality of individual filaments.

As shown in FIG. 2, the distance of overlap 115, 119, 123 between adjacent windings or turns may vary over the length of the elongate shaft 100. As used herein, and as evidenced by the Figures, the distance of overlap may be the distance a winding overlaps the previous winding. For example, as further illustrated in FIG. 4, as the length of material 202 is wound over a mandrel or other rigid device 205, a proximal edge 210 of a subsequent turn 220 may be placed over the preceding adjacent turn 218. The distance 219 between the proximal edge 210 of the subsequent turn 220 and the distal edge 212 (shown in phantom) of the preceding turn 218 is the overlap distance 219. Thus, a proximal portion 214 of a subsequent turn 220 may be disposed, at least in part, over a distal portion 216 of the preceding turn 218. It is contemplated that in some embodiments a subsequent turn 220 may not overlap the preceding turn 218. For example, the proximal edge 210 of the subsequent turn 220 may abut the distal edge 212 of the preceding turn or the proximal edge 210 of the subsequent turn 220 may be spaced a distance from the distal edge 212 of the preceding turn 218. While the overlap distance has been described with reference to two particular turns 218, 220, any two adjacent turns may have any of the above described orientations.

The overlap distance may also be defined as a function of the pitch, or angle, of the helical winding. As used herein, the pitch may be defined as the distance between corresponding points adjacent turns, e.g. from a distal edge of a first winding to a distal edge of an adjacent winding. Thus, a small pitch will have a large distance of overlap whereas a large pitch may not overlap at all, and a zero pitch would result in the length of material being wound perpendicular to the mandrel such at all windings are disposed one on top of the other like a roll of tape. As can be seen, in some embodiments, the pitch, or overlap distance, may be adjusted to cause the catheter shaft 100 to be formed of a single layer of material, two layers of material, three layers, or more. The number of layers forming a given region of the catheter shaft 100 may impact the stiffness of the catheter shaft 100. For example, in some embodiments, a catheter shaft 100 formed from a single layer may be flexible laterally. In other embodiments, a catheter shaft 100 formed from many layers may be extremely rigid. The overlap distance 115, 119, 123 may be adjusted during winding to yield the desired number of layers, and hence the stiffness of the resulting catheter shaft 100. In some embodiments, the stiffness of the catheter shaft 100 may be a function of the cube of the wall thickness and thus may be a function of the cube of the total number of layers. The stiffness of the catheter shaft 100 may also depend on the thickness of each layer, the adhesion between layers, and the material properties of the length of material.

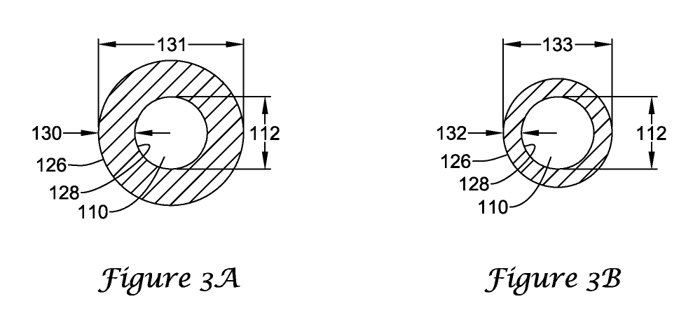

It is further contemplated that the distance of overlap and thus the number of layers of material may vary over the length of the catheter shaft 100. In some embodiments, a proximal portion 104 of the catheter shaft 100 may have first turn 114 having a proximal edge 134 and a distal edge 136 and a second turn 116 having a proximal edge 138 and a distal edge 140, where the distal edges 136,140 are shown in phantom. The proximal edge 138 of the second turn 116 may overlap the distal edge 136 of the first turn 114, defining an overlap distance 115. In some embodiments, spaced a number of turns distal from the first 114 and second 116 turns, the intermediate portion 105 of the catheter shaft 100 may have third turn 118 having a proximal edge 142 and a distal edge 144 and a fourth turn 120 having a proximal edge 146 and a distal edge 148. The proximal edge 146 of the fourth turn 120 may overlap the distal edge 144 of the third turn 118, defining an overlap distance 119. In some embodiments, spaced a number of turns distal from the third 118 and fourth 120 turns, the distal portion 106 of the catheter shaft 100 may have fifth turn 122 having a proximal edge 150 and a distal edge 152 and a sixth turn 124 having a proximal edge 154 and a distal edge 156. The proximal edge 154 of the sixth turn 124 may overlap the distal edge 152 of the fifth turn 122, defining an overlap distance 123. Thus, in some embodiments, the catheter shaft 100 may have a relatively large overlap distance 115 at a proximal portion 104, a moderate overlap distance 119 in the intermediate portion 105, and a short overlap distance 123 at the distal portion 106. The overlap distances 115, 119, 123 illustrated in the proximal 104, intermediate 105, and distal 106 portions are not intended to be limiting, merely illustrative of how the overlap distance may change and impact the catheter properties, as discussed in more detail with respect to FIGS. 3A and 3B. Furthermore, while the overlap distances 115, 119, 123 are illustrated as getting progressively smaller from the proximal end 104 to the distal end 106, it is contemplated that the overlap distance may vary along the length as desired based on the desired characteristics of the catheter shaft. For example, the overlap distance or layers of winding may be made to increase substantially in a region where the catheter shaft is joined to a hub or adaptor to provide built in strain relief.

The resulting catheter shaft 100 may have a wall thickness that changes dynamically and/or continuously along the length of the catheter shaft 100 in proportion to the change in overlap distance. Accordingly, the stiffness of the catheter shaft 100 may also change dynamically along the length of the catheter shaft 100. While the catheter shaft 100 is illustrated as having a continuously changing overlap distance 115, 119, 123, is contemplated that, in some embodiments, the overlap distance may vary in a step-wise manner. For example, the proximal portion 104 may have a first thickness, and thus a first overlap distance, the intermediate portion 105 may have a second thickness, and thus a second overlap distance, different than the first thickness, and the distal portion 106 may have a third thickness, and thus a third overlap distance, different than the second thickness, such that the profile of the outer diameter of the catheter shaft 100 may resemble a set of stairs. In some embodiments, the overlap distance may be varied, either continuously or step-wise, to generate a catheter shaft having variable flexibility along the length thereof in any fashion desired. For example, the catheter shaft 100 may be constructed to have a proximal region of the first stiffness, an intermediate region of the second stiffness and a distal region having the same stiffness as the proximal region.

FIGS. 3A and 3B are illustrative cross-sections of the illustrative catheter shaft 100 of FIG. 2 taken at lines 3A-3A and 3B-3B, further demonstrating how the overlap distance may affect the wall thickness. FIG. 3A is a representative cross-section of a proximal end region 104 of the catheter shaft 100. The catheter shaft 100 may have an outer surface 126 and an inner surface 128 defining a wall thickness 130 there between. FIG. 3B is a representative cross-section of a distal end region 106 of the catheter shaft 100. The catheter shaft 100 may have an outer surface 126 and an inner surface 128 defining a wall thickness 132 there between. In some embodiments, the wall thickness 130 of the proximal end region 104 may be larger than the wall thickness 132 of the distal end region 106. This may correspond to the larger overlap distance 115 of the proximal end region 104 compared to the smaller overlap distance 123 of the distal end region 106. In some embodiments, the inner diameter 112 of the tubular member 102 may remain constant from the proximal end 103 to the distal end 106 of the catheter shaft 100. In some embodiments, as the wall thickness of the catheter shaft 100 varies from the proximal end region 104 to the distal end region 106, the outer diameter of the catheter shaft may vary over the length of the catheter shaft 100 as well. For example, the outer diameter 131 of the proximal end region 104 may be larger than the outer diameter 133 of the distal end region 106. In some embodiments, the outer diameter of the catheter shaft 100 may taper from proximal end region 104 to the distal end region 106 when the overlap distance is continuously varied as shown in FIG. 2. However, in other embodiments, if the overlap distance is varied in a step-wise manner, the catheter shaft 100 may have regions of uniform diameter, separated from one another by abrupt changes in diameter (e.g. as in stairs). It is contemplated that the outer diameter or wall thickness of the catheter shaft 100 may vary as a function of the desired stiffness in any way desired. For example, in some embodiments, while not explicitly shown, the proximal end region 104 may have a smaller outer diameter than the distal end region 106. While the cross-sections of the catheter shaft 100 of FIGS. 3A and 3B are shown as having a generally circular cross-section, it is contemplated the catheter shaft 100 may have any cross-sectional shape desired. For example, the catheter shaft 100 may be out of round such that it has a preferred bending plane.

An abrupt change in the pitch of the winding, as may be required for a step-wise change in overlap distance, may be difficult to accomplish if the length of material 202 is stiff. While not explicitly shown, in some embodiments, a cut placed perpendicular to the edge 210, 212 of the length of one material or a U-shaped notch (also perpendicular to the edge 210, 212) may be periodically provided along one or both edges 210, 212 or alternating edges of the length of material 202 to allow the length of material to stretch, at least in part, when the pitch first changes. It is contemplated that if these cuts are not too close together they may have little effect on the stiffness of the catheter shaft 200.

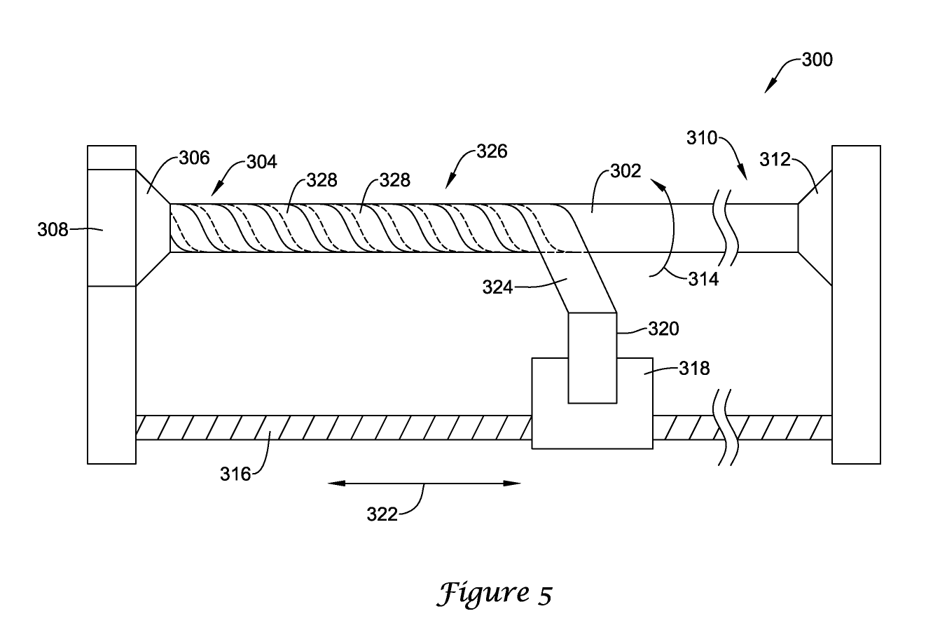

Turning to FIG. 5, an illustrative method of manufacturing a variable flexibility catheter shaft 100, such as shown in FIG. 2, will now be described. In some embodiments, a manufacturing assembly 300 may be provided to facilitate the manufacture of the catheter shaft 326. A mandrel 302 may be provided as a first part of the manufacturing assembly 300. A first end 304 of the mandrel 302 may be positioned within a chuck 306 or other suitable mounting device. The chuck 306, in turn, may be rotationally connected to a lathe 308 or other suitable driving device. A second end 310 of the mandrel 302 may be positioned within a tailstock 312, or other suitable mounting device. The lathe 308 may be configured to impart rotational movement 314 on the mandrel 302. As a second part of the manufacturing assembly 300, a carriage assembly 318 carrying a spool 320 of material 324 may be provided adjacent to the mandrel 302. The carriage assembly 318 may move longitudinally 322 along the length of the mandrel 302 on a lead screw 316, or other suitable track mechanism.

The length of material 324 may be helically wrapped around the mandrel 302 beginning at either the first 304 or second end 310, or anywhere there between, of the mandrel 302. For example, the carriage assembly 318, and hence the spool 320 of material 324, may be initially located laterally adjacent to the first end 304 of the mandrel 302. An end of the length of material 324 may be secured to the mandrel 302 and rotational movement 314 of the mandrel 302 may begin. As the mandrel 302 rotates, the carriage assembly 318 may move longitudinally along the length of the mandrel 302 from the first end 304 to the second end 310. The rotation 314 of the mandrel 302 may cause the length of material 324 to be transferred from the spool 320 to the mandrel 302 resulting in a plurality of turns 328 forming a catheter shaft 326. In an alternative embodiment, the mandrel 302 may remain still while a spool 320 of material 324 is moved around the circumference of the mandrel 302 resulting in a plurality of turns 328. In some embodiments, the inner diameter of the catheter shaft 326 may be tapered using a tapered mandrel 302 and varying where on the mandrel 302 the material is wrapped. It is contemplated that in some instances, the newly formed catheter shaft 326 may be drawn off the end of the mandrel 302 as the catheter shaft 326 is formed.

As discussed above, the length of material may include an adhesive on the inner surface 211 (see FIG. 4) the material. In some embodiments, the adhesive may be included over the entire inner surface 211, whereas in other embodiments, the adhesive may included over a portion of the inner surface 211. For example, the adhesive may be included on only the proximal edge region 214 of the length of material. The adhesive may help secure a subsequent turn to the adjacent preceding turn, which may result in a stiffer catheter shaft 326. In some instances, the adhesive may be a pressure sensitive adhesive. Prior to removing the catheter shaft 326 from the mandrel 302, pressure may be applied to the outer surface of the catheter shaft 326 to activate a pressure sensitive adhesive. In other embodiments, the catheter shaft 326 may be heated, while still on the mandrel 302, just enough to cause the adhesive on the overlapping turns to flow. The adhesive may be sufficient to adequately bind the overlapping turns 328 to create a fluid tight seal along the length of the catheter shaft 326. In the absence of an adhesive, the catheter shaft 326 may be heated, while still on the mandrel 302, just enough to cause the overlapping turns to flow. The catheter shaft 326 may then be cooled such that the material hardens (as in a thermoplastic polymer) resulting in a fluid tight seal along the length of the catheter shaft. When the layers (overlap distance) are bonded together, by adhesive or otherwise, the resulting catheter shaft 326 may have good torque response. After the overlapping turns have been bonded, if desired, the resulting catheter shaft 326 may be removed from the mandrel. In some embodiments, the final product may be a tubular member similar to tubular member 102 illustrated in FIG. 2.

In some embodiments, it may be desirable to impart an additional shape to the newly wound catheter shaft 326. For example, in some instances, it may be desirable for the distal end of the catheter shaft 326 to have a curved shape. A newly wound catheter may be placed over a mandrel having the desired shape, such as a “J” shape to impart a curve on the distal end. Once the catheter shaft 326 has been placed over the mandrel, heat or pressure may be applied to activate an adhesive. Once the adhesive has been set, the catheter shaft 326 having the curved shape may be removed from the mandrel. While the catheter shaft 326 is described as having a curved distal tip, it is contemplated that the catheter shaft 326 may be formed having any shape desired at any desired location along the length of the catheter shaft.

It is contemplated that the overlap distance of adjacent turns may be varied by varying the speed at which the mandrel 302 rotates or by adjusting the speed at which the carriage assembly 318 moves longitudinally along the length of the mandrel 302, or both. For example, the faster the mandrel 302 rotates, the greater the distance of overlap may be, resulting in stiffer catheter shaft. A slowly rotating mandrel 302 may result in a small overlap distance or even no overlap at all, resulting in a less stiff, or more flexible, catheter shaft 326. As way of further example, the slower the carriage assembly 318 moves, the greater the distance of overlap may be. Whereas, a faster moving carriage assembly 318 may result in a small overlap distance or even no overlap at all. As can be seen, both the speed of rotation of the mandrel 302 and the speed of longitudinal movement of the carriage assembly 318 may be manipulated to vary the overlap distance between adjacent turns. If the speed of either the mandrel 302 of the carriage assembly 318 is increased or decreased at a constant rate, the overlap distance between adjacent turns may vary continually over the length of the catheter shaft 326, e.g. the overlap distance may be slightly different for each set of adjacent turns. As can be seen, this may result in a catheter shaft with continuously changing stiffness.

As by way of further example, the speed of the components of the manufacturing assembly 300 may be configured such that a proximal portion of the catheter shaft 326 is stiffer than a distal portion. In some embodiments, it may be desirable for a given portion of the catheter shaft 326 to have the same stiffness, and thus the given portion may have an overlap distance between adjacent turns that is the same or very similar. In this instance, the manufacturing assembly 300 may be caused to move at a constant speed along the length of the mandrel 302 corresponding to the desired region of uniform stiffness of the catheter shaft 326. It is further contemplated that in some embodiments, some catheter shafts 326 may be manufactured having a combination of continuously varied regions of stiffness and constant (or approximately constant) regions of stiffness.

In some embodiments, it may be desirable to manufacture a catheter shaft 326 using more than one length of material 324. For example, a first length of material 324 may be wrapped around the mandrel 302 using any of the above described methods to achieve a first layer of material. It is contemplated that adjacent turns of the first layer of material may have little, if any, overlap, if desired. A second length of material may be subsequently wrapped over the first layer of material using any of the above described methods to achieve a second layer. Again, it is contemplated adjacent turns of the second layer may have little, if any, overlap, if desired. The second length of material may be formed from a material different than the first length of material or from the same material. This process may be repeated for any number of layers desired using all the same material or a combination of materials. The number of layers may be chosen based on the desired stiffness of the catheter shaft 326. In some embodiments, the second layer, or further subsequent layers, may be wrapped in the same helical direction as the first layer. In other embodiments, the second layer, or further subsequent layers, may be wrapped in a direction opposite the first layer. It is contemplated any of the layers may be wrapped in any direction based on the desired properties of the catheter shaft 326. In the event a second layer is wrapped in a direction opposite the first layer, the resulting catheter shaft 326 may have enhanced torque control. It is further contemplated that the second layer, or further subsequent layers, may not extend over the entire length of the catheter shaft 326. For example, a second layer may begin at the proximal end region of the catheter shaft and may terminate at a location proximal the distal end of the catheter shaft. As by way of further example, in some embodiments, an additional layer may be disposed over the intermediate region of the catheter shaft. In some embodiments, the second, or further subsequent layers, may be wrapped from a different starting location than the first or preceding layer.

It is contemplated that more than one type of material may be used to form the helically wound catheter shaft 326. For example, in some embodiments, it may be desirable to use a stiffer length of material 324 for a first portion of the catheter shaft and a more flexible length of material 324 for a second portion of the catheter shaft. In some embodiments, the materials may be chosen such that the stiffness of the catheter may be varied while maintaining a constant or relatively constant inner and outer diameter. In some instances, a distal end of the catheter shaft 326 may be formed from a material configured to provide the catheter shaft 326 with an atraumatic tip. For example, the distal end may be formed from a material softer than the material used to form the proximal end.

In other embodiments, more than one length of material may be wrapped around the mandrel 302 simultaneously. For example the manufacturing assembly 300 may include more than one carriage assembly 318 for wrapping more than one length of material 324 at the same time. In this embodiment, a second carriage housing may be disposed adjacent to a first carriage assembly 318 or the second carriage housing may be disposed at a distance from the first carriage assembly 318. The second carriage assembly may move longitudinally along the length of the mandrel 302 following behind the first carriage assembly 318, such that two lengths of material are wrapped around the mandrel 302 simultaneously.

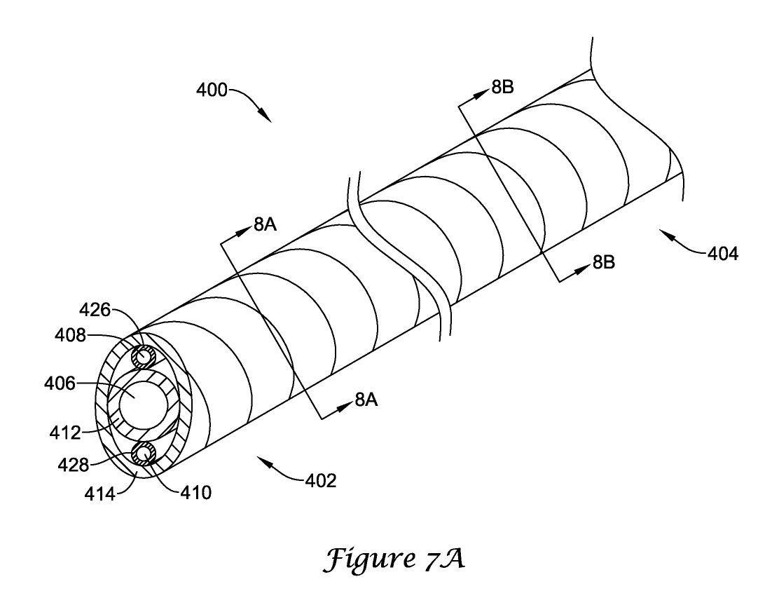

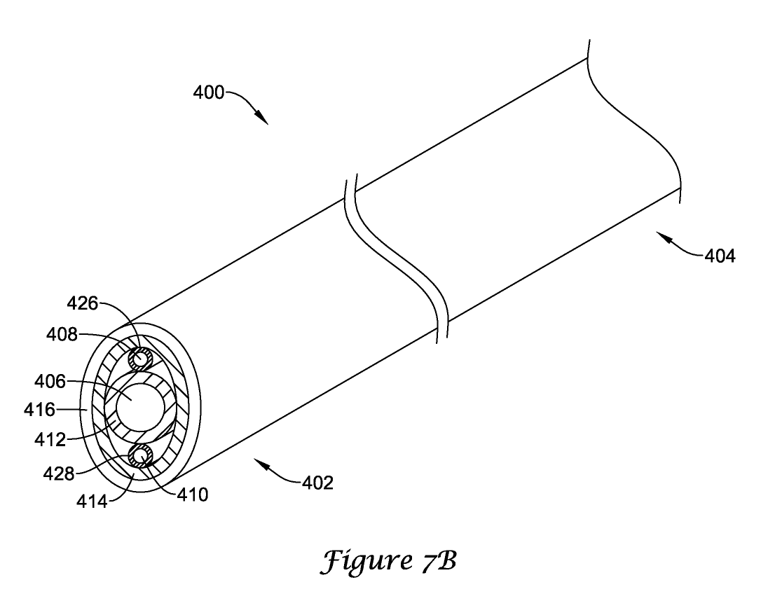

In some embodiments, a thin walled tube (not explicitly shown) may be disposed over the mandrel 302 prior to forming the catheter shaft 326. The thin walled tube may form the inner surface of the final catheter shaft 326. The thin walled tube may allow the catheter shaft 326 to have a smooth inner surface to facilitate the advancement of additional medical devices or treatments within the lumen. Alternatively, the thin walled tube may be removed after the catheter shaft 326 has been formed to allow control over the inner and outer wall properties of the resulting catheter shaft 326. For example, the thin walled tube may be placed along the mandrel 302 in a region corresponding to a desired region of reduced wall thickness. This may help maintain a relatively constant outer diameter in the final catheter shaft 326 while still allowing a varying wall thickness to control the stiffness. Further, as discussed in more detail with respect to FIG. 7B, a polymer sheath may be disposed over the helically wrapped catheter shaft 326 to make a smooth outer surface or to impart additional stiffness. In other embodiments, the mandrel 302 may comprise a central continuous thin walled tube. In order to provide the additional support to the thin walled tube while the length of material 324 is wrapped around the tube, sterile water may be pumped through the thin walled tube and frozen just prior to the wrapping. Once the catheter shaft 326 has been formed, the ice may be melted and the water removed.

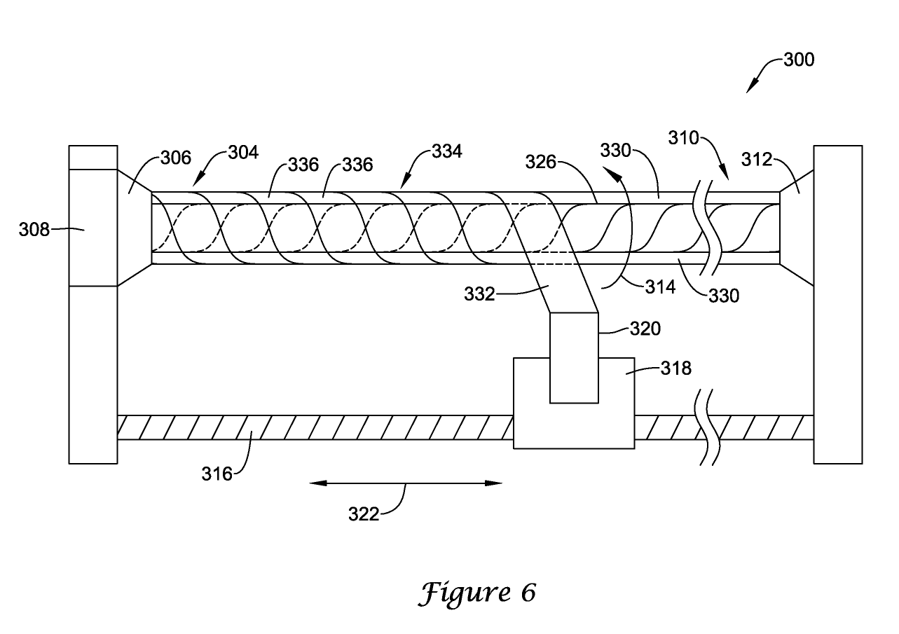

In some embodiments, it may be desirable for the illustrative catheter shaft 100 to have more than one lumen. Turning to FIG. 6, an illustrative method of manufacturing a multi-lumen variable flexibility catheter shaft 334 will now be described. A single lumen catheter shaft 326 formed using any of the above described methods may remain on the mandrel 302 or may be removed from the mandrel 302 as desired. One or more additional mandrels 330 may be placed adjacent to the catheter shaft 326. It is contemplated that any number of additional mandrels 330 may be used to form a catheter shaft 334 having the desired number of lumens, for example, but not limited to, one, two, three, four or more additional mandrels. While the two additional mandrels 330 shown in FIG. 6 are placed opposite one another, it is contemplated the additional mandrels 330 may be placed in any orientation desired. As with the single lumen catheter shaft described above, the one or more additional mandrels 330 may include thin walled tube (not explicitly shown) disposed over the mandrel(s) 330 prior to forming the multi-lumen catheter shaft 334. Alternatively, it is contemplated that in some embodiments, thin walled tubes may be used in place of a mandrel. Thin walled tubes may remain in the catheter shaft 326 to become lumens in the catheter.

A second length of material 332 may be helically wrapped around the mandrels 330 and the single lumen catheter shaft 326 beginning at either the first or second end 304, 310, or anywhere there between. For example, the carriage assembly 318, and hence the spool of material 320, may be initially located laterally adjacent to the first end 304 of the mandrels 302, 330. An end of the length of material 332 may be secured to the mandrels 330 or the single lumen catheter shaft 326 and rotational movement 314 of the mandrels 302, 330 may begin. As the mandrels 302, 330 rotate, the carriage assembly 318 may move longitudinally along the length of the mandrels 302, 330 from the first end 304 to the second end 310. The rotation 314 of the mandrel 302 may cause the length of material 332 to be transferred from the spool 320 to the mandrels 330 and the single lumen catheter shaft 326 resulting in a plurality of turns 336 forming a multi-lumen catheter shaft 334. In an alternative embodiment, the mandrels 302, 330 may remain stationary while a spool 320 of a length of material 332 is moved around the circumference of the mandrels 302, 330 resulting in a plurality of turns 336. As discussed above, it is contemplated that the overlap distance of adjacent turns, the number of layers and helical direction of the layers may be adjusted based on the desired properties of the final catheter shaft 334. Further, the length of material 332 may be formed from a material different than the first length of material 324 or from the same material as the first length of material 324 depending on the desired properties of the final catheter shaft 334.

FIGS. 7A and 7B show an illustrative multi-lumen catheter shaft 400 formed in accordance with the method described above. The multi-lumen catheter shaft 400 may have a proximal end region 402 and a distal end region 404 with an intermediate region disposed there between (not explicitly shown). In some embodiments, the multi-lumen catheter shaft 400 may have a first lumen 406 defined by an inner tubular member 412. In some embodiments, the inner tubular member 412 may be formed of a length of material having a plurality of turns as described with respect to FIGS. 2 and 4. In other embodiments, the inner tubular member 412 may be an extruded tubular member or other preformed tubular member known in the art. In some embodiments, the multi-lumen catheter shaft 400 may further include a second and a third lumen 408, 410. The second and third lumens 408, 410 may be defined by thin walled tubes 426, 428 which may have been disposed over the mandrels prior to forming the catheter shaft 400. In the absence of thin walled tubes 426, 428, the second and third lumens 408, 410 may be the space between the helically wound outer member 414 and the inner tubular member 412. In some embodiments, the second and third lumens 408, 410 may have a generally crescent shape. However, it is contemplated the second and third lumens 408, 410 may have any shaped desired based on the mandrels used.

As illustrated in FIG. 7B, in some embodiments, the catheter shaft 400 may further include a polymer sheath 416 disposed over the helically wound layers. While not explicitly shown, it is contemplated that inner tubular member 412 or single lumen catheter shaft 100 may also include a polymer sheath disposed over the helically wound layer. The polymer sheath 416 may be formed over the helically wound layer 414 (or other layer) in any method desired, for example, but not limited to: extrusion, heat shrinking, dipping, powder coating, etc. The polymer sheath 416 may provide a smooth outer surface which may reduce the likelihood of clot formation on the catheter shaft 400. The polymer sheath may be formed of any material desired such as, but not limited to polyether block amides (such as Pebax®, manufactured by Arkema), urethanes, polyurethanes, polyamides (such as nylons) etc. In some embodiments, the polymer sheath may further include a lubricious or hydrophilic coating on the catheter shaft, such as, but not limited to polytetrafluoroethylene (PTFE).

As with the single lumen catheter 100 discussed above, it is contemplated that the number of layers of material may vary over the length of the catheter shaft 400 for both the inner member 412 and the outer member 414. For example, the overlap distance may vary continuously from a proximal portion 402 to a distal portion 404 of the catheter shaft 400. The resulting catheter shaft 400 may have a wall thickness or wall thicknesses that change dynamically along the length of the catheter shaft 400 in accordance with the change in overlap distance. Accordingly, the stiffness of the catheter shaft 400 may also change dynamically along the length of the catheter shaft 400. As discussed above, the overlap distance, thickness, number of layers, and/or types of materials may be chosen based on the desired properties of the final catheter shaft 400.

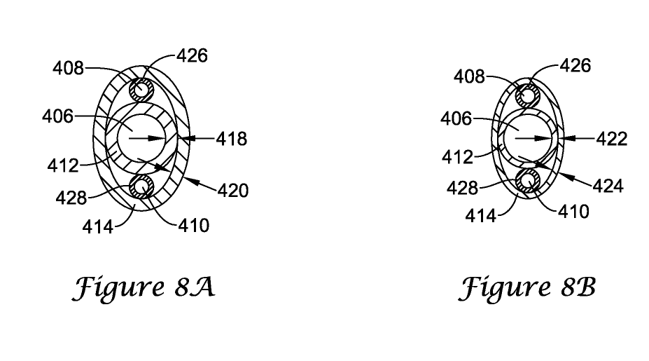

FIGS. 8A and 8B are illustrative cross-sections of the illustrative catheter shaft 400 of FIG. 7A taken at lines 8A-8A and 8B-8B, further demonstrating how the overlap distance may affect the wall thickness. FIG. 8A represents an illustrative cross-section of a proximal end region 402 of the catheter shaft 400. The inner tubular member 412 may have a first wall thickness 418 and the outer member 414 may also have a first wall thickness 420. While the wall thicknesses 418, 420 are illustrated as being similar for both the inner member 412 and the outer member 414, it is contemplated that either wall thickness may be varied as desired, e.g. one may be thicker or thinner than the other. FIG. 8B represents an illustrative cross-section of a distal end region 404 of the catheter shaft 400. The inner tubular member 412 may have a second wall thickness 422 and the outer member 424 may also have a second wall thickness 420. While the wall thicknesses 422, 424 are illustrated as being similar for both the inner member 412 and the outer member 414, it is contemplated that either wall thickness may be varied as desired, e.g. one may be thicker or thinner than the other. The wall thicknesses 418, 420 of the proximal end region 402 may be larger than the wall thicknesses 422, 424 of the distal end region 404. This may correspond to a larger overlap distance of the proximal end region 402 (and hence stiffer) compared to a smaller overlap distance of the distal end region 404 or to more layers. The diameters of lumens 406, 408, 410 may remain constant from the proximal end 402 to the distal end 404 of the catheter shaft 400. However, the cross-sectional area of the outer member 414 may taper from the proximal end 402 to the distal end 404 as a result of the reduced outer diameter of the inner tubular member 412. As one can see, for this particular embodiment, this may result in the outer profile of the catheter shaft 400 varying over the length of the catheter shaft 400. However, in other embodiments, the outer diameter or wall thickness tubular members 412, 414 and the overall profile of the catheter shaft 400 may vary as a function of the desired stiffness in any way desired. For example, in some embodiments, the proximal end region 402 may have a smaller outer profile than the distal end region 404.

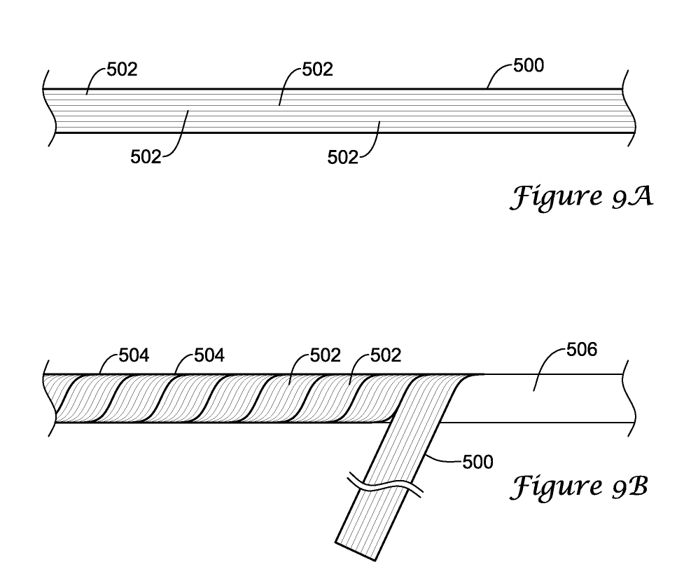

In some embodiments, the length of material may comprise a composite material. For example, as illustrated in FIGS. 9A and 9B, the length of material 500 may include one or more reinforcing filaments 502 extending along the length thereof. While the present embodiment is illustrated as having seven reinforcing filaments 502, it is contemplated the length of material 500 may have any number of reinforcing filaments 502 desired, such as, but not limited to 1, 2, 4, 8 or more, or any number there between. The reinforcing filaments 502 may be comprised of a material configured to provide additional circumferential or axial strength to the length of material 500. For example, the reinforcing filaments 502 may include, but are not limited to stainless steel, superelastic metal alloys, high strength polymers (such as Kevlar®, manufactured by DuPont), etc. In some embodiments, the filaments 502 may be electrically conductive so as to relay electrical signals or energy to a treatment device. As illustrated in FIG. 9B, as the length of material 500 is wound around a mandrel 506, creating a plurality of turns 504, the reinforcing filaments 502 are also wrapped around the mandrel 506. The reinforcing filaments 502 may provide additional strength to the final catheter shaft. In the event that a second layer of material including the reinforcing filaments 502 is wrapped in a direction opposite the first layer the filaments 502 of the second layer may cross the filaments 502 of the first layer. This orientation may provide enhanced torque control in the resulting catheter shaft. It is further contemplated the number of filaments 502, orientation of the filaments 502, and the number of layers including filaments 502 may be varied according to the desired properties of the catheter shaft.

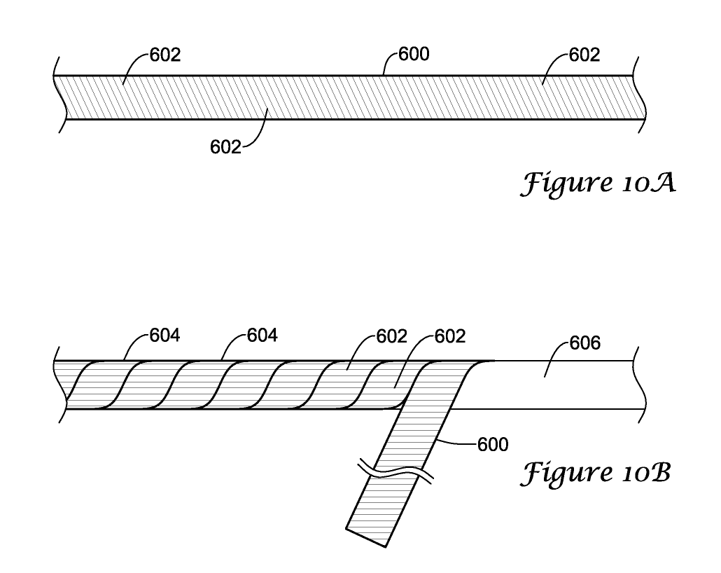

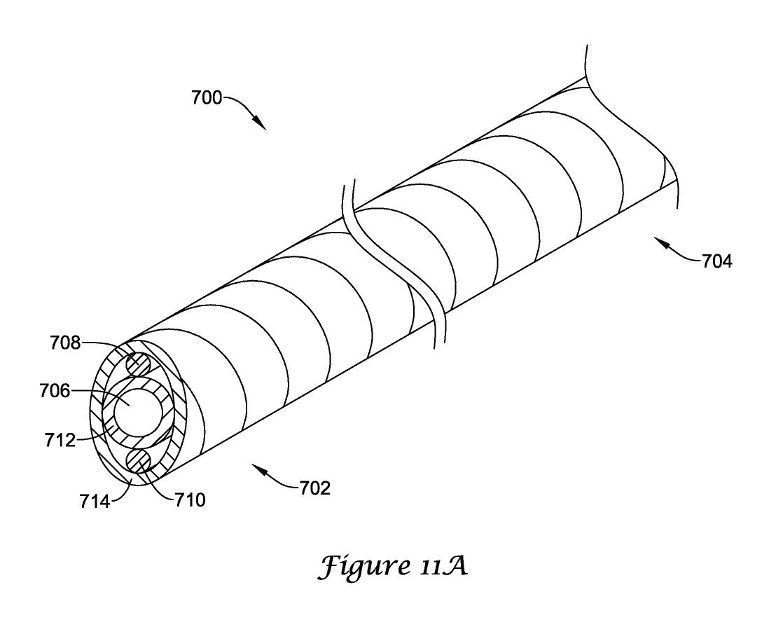

In other embodiments, such as shown in FIGS. 10A and 10B, the length of material 600 may include one or more short filament segments 602 extending at an angle to a longitudinal axis of the length of material 600. The angle of the filaments 602 may be chosen such that when the length of material 600 is wrapped around the mandrel 606 to define a plurality of turns 604, the filaments 602 may be oriented parallel to the longitudinal axis. However, it is contemplated that the filaments may be oriented at an angle desired. The alignment of the filaments 602 along the longitudinal axis may provide flexural stiffness and greater tensile strength to the resulting catheter shaft. While the filaments 602 are shown as aligning when the length of material 600 is wrapped around the mandrel 606, it is contemplated the filaments 602 may not align along the length of the catheter shaft. Further, while the length of material 600 is shown as having many filaments 602, the length of material 600 may have any number of filaments 602 desired, such as, but not limited to 1, 2, 4, 8 or more, or any number there between. The reinforcing filaments 602 may be comprised of a material configured to provide additional strength to the length of material 600. For example, the reinforcing filaments 602 may include, but are not limited to stainless steel, superelastic metal alloys, high strength polymers, etc. In some embodiments, the filaments 602 may be electrically conductive so as to relay electrical signals or energy to a treatment device. It is further contemplated the number of filaments 602, orientation of the filaments 602, and the number of layers including filaments 602 may be varied according to the desired properties of the catheter shaft.

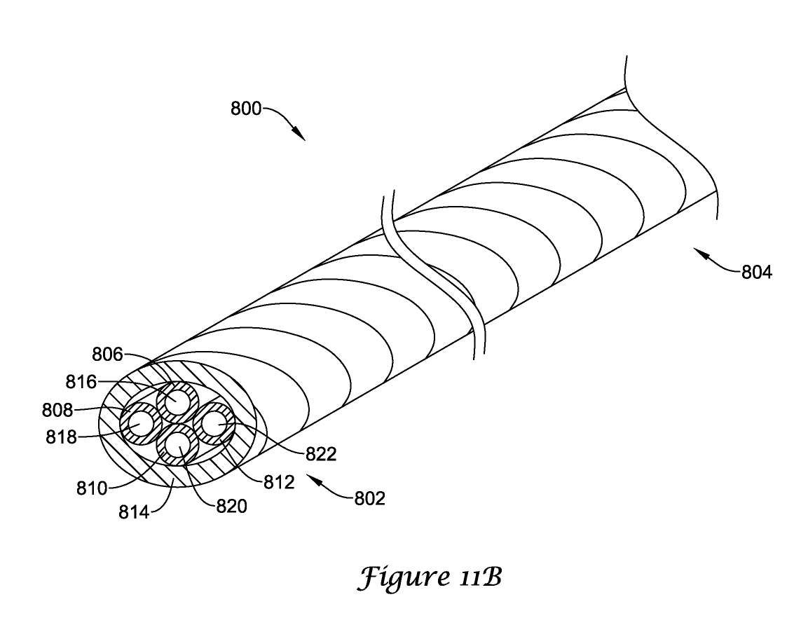

FIG. 11A is a perspective view of an alternative embodiment of an illustrative catheter shaft having a helically wrapped shaft 700 including a proximal portion 702 and a distal portion 704. In some instances, the catheter shaft 700 may include a number of thin metal or plastic stiffening wires 708, 710 aligned around the circumference and extending parallel to central tubular member 712 to provide varying stiffness along the length of the catheter shaft 700. For example, the wires 708, 710 may vary in length to provide varying stiffness along the length of the final catheter shaft 700. In other embodiments, the wires may be helically or circumferentially wound to impart kink resistance to the catheter shaft 326. In other embodiments, the wires 708, 710 may be shaping ribbons or braids may be used to provide varying stiffness along the length of the final catheter shaft. It is further contemplated the wires 708, 710 may include a number of electrical wires extending along the length of the catheter shaft 700 so as to relay electrical signals or energy to a treatment device. In some embodiments, central tubular member 712 may be a helically wrapped elongate tubular member defining a lumen 706 therethrough. In other embodiments, central tubular member 712 may be an extruded, or otherwise preformed, tubular member. It is contemplated that in some embodiments, the wires 708, 710 may be positioned under a helically wound layer, such as layer 708. While not explicitly shown, in some instances, a thin walled tube may be heat shrunk or extruded over the wires 708, 710 to provide a jacket over the wires 708, 710.

FIG. 11B is a perspective view of an alternative embodiment of an illustrative multi-lumen catheter shaft 800 having a helically wrapped shaft including a proximal portion 802 and a distal portion 804. In some embodiments, a multi-lumen catheter shaft 800 may be formed by clustering a number of thin walled tubes 806, 808, 810, 812 to form a bundle. The thin walled tubes 806, 808, 810, 812 may each define a lumen 816, 818, 820, 822 therethrough, thus defining a catheter shaft 800 having at least four lumens. In some instances, the bundle may be used in place of, or in combination with, the first and second mandrels 302, 330. The length of material may be wrapped around the outer surfaces of the bundle in a helical manner such as that described above, forming outer layer 814. The length of material may bind the bundle together in addition to imparting additional stiffness to the multi-lumen catheter shaft 800. In some embodiments, one or more of the thin walled tubes 806, 808, 810, 812 forming the bundle may have different inner diameters and/or wall thicknesses as desired such that the multi-lumen catheter shaft 800 may have lumens 816, 818, 820, 822 with varying sizes (not explicitly shown). Alternatively, in other embodiments, one or more of the thin walled tubes 806, 808, 810, 812 forming the bundle may have the same dimensions. In some embodiments, the thin walled tubes 806, 808, 810, 812 may each be formed from the same material. In other embodiments, one or more thin walled tubes 806, 808, 810, 812 may be formed from a different material than the others as desired. While the illustrative embodiment is shown having a cluster of four thin walled tubes 806, 808, 810, 812, it is contemplated the multi-lumen catheter shaft 800 may have an number of tubes desired, such as, but not limited to, two, three, five, or more.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A medical device comprising:
a tubular member having a proximal end and a distal end, the tubular member including a length of material helically wrapped defining a plurality of turns, the length of material having a width including a first edge and a second edge; and
wherein at least some of the turns are wrapped in an overlapping fashion such that at least a portion of a first edge of a turn overlaps at least of portion of a second edge of a previous turn defining an overlap distance.

2. The medical device of claim 1, wherein a proximal portion of the tubular member includes turns wrapped in an overlapping fashion that have a first overlap distance and a distal portion of the tubular member includes turns wrapped in an overlapping fashion that have a second overlap distance.

3. The medical device of claim 1, wherein the overlap distance varies from the proximal end of the tubular member to the distal end of the tubular member.

4. The medical device of claim 2, wherein the first overlap distance is greater than the second overlap distance.

5. The medical device of claim 1, wherein the stiffness of the tubular member varies from the proximal end to the distal end.

6. The medical device of claim 1, wherein the distal end is more flexible than the proximal end.

7. The medical device of claim 1, wherein the length of material further comprises a plurality of filaments extending parallel to a longitudinal axis of the length of material.

8. The medical device of claim 1 further comprising a second tubular member disposed over the tubular member.

9. The medical device of claim 1, wherein the length of material further includes a first side and a second side positioned opposite the first side, wherein at least a portion of the first side includes an adhesive.

10. The medical device of claim 9, wherein the adhesive is a pressure sensitive adhesive.

11. A catheter shaft comprising:
a tubular member having a proximal end and a distal end, the tubular member including a length of material helically wrapped defining a plurality of turns, the length of material having a width including a first edge and a second edge;
wherein the plurality of turns includes a first turn and a second turn adjacent to the first turn, wherein at least a portion of the first edge of the second turn overlaps at least of portion of the second edge of the first turn defining a first overlap distance; and
wherein the plurality of turns includes a third turn and a fourth turn adjacent to the third turn, wherein at least a portion of the first edge of the fourth turn overlaps at least of portion of the second edge of the third turn defining a second overlap distance.

12. The catheter shaft of claim 11, wherein the third and fourth turns are spaced a number of turns distal from the first and second turns.

13. The catheter shaft of claim 11, wherein the second overlap distance is smaller than the first overlap distance.

14. The catheter shaft of claim 11, wherein the length of material comprises a polymer.

15. A method of manufacturing a medical device, the method comprising:
providing a mandrel having a longitudinal axis, a first end and a second end;
providing a length of material having a width including a first edge and a second edge, wherein the length is larger than the width;
wrapping the length of material around the mandrel in a helical manner, the length of material defining a plurality of turns;
removing the mandrel such that the plurality of turns defines a tubular member including a lumen;
wherein for at least some of the turns at least a portion of the first edge of a turn overlaps at least of portion of the second edge of a previous turn.

17. The method of claim 16, wherein wrapping the length of material around the mandrel includes wrapping a first number of turns near the first end of the mandrel such that the first number of turns has a first overlap distance and wrapping a second number of turns near the second end of the mandrel such that the second number of turns has a second overlap distance

18. The method of claim 17, wherein the overlap distance of the plurality of turns varies from the first end of the mandrel to the second end of the mandrel.

19. The method of claim 15, wherein the first length of material is wrapped in a first direction, further comprising:
providing a second length of material having a width including a first edge and a second edge, wherein the length is larger than the width; and
winding the second length of material around the mandrel in a second direction opposite the first direction, the second length of material defining a plurality of turns;
wherein at least a portion of the first edge of a turn of the second length of material overlaps at least of portion of the second edge of a previous turn of the second length of material.

20. The method of claim 15, further comprising:
providing one or more secondary mandrels;
positioning the one or more secondary mandrels adjacent to the tubular member;
providing a second length of material having a width including a first edge and a second edge, wherein the length is larger than the width; and
winding the second length of material around the mandrels, the second length of material defining a plurality of turns;
wherein at least a portion of the first edge of a turn of the second length of material overlaps at least of portion of the second edge of a previous turn of the second length of material.
